(19) United States
(12) Patent Application Publication
(10) Pub. No.: US 2012/0123389 A1
(43) Pub. Date: May 17, 2012

(54) Adjustable Length Access Sheath

(76) Inventor: David Shafran, Richmond, KY (US)

(21) Appl. No.: 12/945,130

(22) Filed: Nov. 12, 2010

(51) Int. Cl. A61B 1/32

(52) U.S. Cl. 604/523

(57) ABSTRACT

An adjustable length sheath and a method for adjusting the length of a sheath is disclosed. The adjustable length sheath is primarily designed for uretral access, and for the insertion of a dilator or other medical equipment. The adjustable length sheath has a proximal end with circumferential grooves cut lateral to the axis of said sheath such that it is possible to snap off a portion of the proximal end to adjust the length of the sheath. The proximal end may have a lower degree of flexibility than the main body of the sheath to allow the end to be more easily broken and may be made from a different, less flexible material, than the main body of the sheath.

### CROSS REFERENCE TO RELATED APPLICATIONS

Not Applicable

### STATEMENT REGARDING FEDERALLY SPONSORED RESEARCH OR DEVELOPMENT

Not Applicable

### THE NAMES OF THE PARTIES TO A JOINT RESEARCH AGREEMENT

Not Applicable

### INCORPORATION-BY-REFERENCE OF MATERIAL SUBMITTED ON A COMPACT DISC

Not Applicable

### BACKGROUND OF THE INVENTION

1. Field of the Invention

The present invention relates generally to an adjustable length guide sheath for ureteral access, and more specifically to a means for adjusting the length of the sheath that extends from the urethra after insertion.

2. Description of the Related Art

The use of catheters, guide-way tubing, and access sheaths for insertion of medial instrumentation into the human body is well known. Examples of prior art patents disclosing various sheathing, catheters, endoscopes, and tubing include U.S. Pat. No. 4,323,071, U.S. Pat. No. 4,569,347, U.S. Pat. No. 5,380,304, U.S. Pat. No. 5,700,253, U.S. Pat. No. 5, 919, 170, and U.S. Pat. No. 7,135,015. U.S. Pat. No. 5,919,170 to Woessner, describes a common method of inserting a catheter into the urinary tract, and the insertion of sheaths and other medical equipment follows essentially the same procedure. Typically there are two co-axially disposed elongated members that are inserted into the body through a vein or the urinary tract. There is an external access sheath, and the internal surgical equipment, such as an endoscope, obturator or dilator which is disposed within the central bore of the sheath. Once the access sheath is in the proper position within the body, a variety of medical equipment can be run through the central bore of the sheath to perform a variety of medical procedures within the body.

A common problem encountered by the prior art is in determining the proper length of the sheath. During certain medial procedures, for example the removal of kidney stones, the sheath will run through the urinary tract and into the kidney to be position near the kidney stone. In this procedure the surgeon passes a ureteroscope through the urinary tract and positions it within the ureter at a point close to the stone, whether the stone is lodged in the ureter or kidney. In some cases the stone can be removed by a forceps passed through the sheath, in other cases the stone is broken up by use of a laser or sonar. In all cases it is important for the end of the sheath to be near the kidney stone for the medical procedure to be properly accomplished. If the sheath is too short it will not be positioned close enough to the kidney stone to be effective, and the sheath will have to be removed and a new longer sheath reinserted. This has the potential to cause damage in the patient's urinary tract, and should be avoided if possible. If the sheath is too long it will require either cutting the protruding end of the sheath to the appropriate length, or require the surgeon to manipulate delicate and sensitive medical equipment away from the urethral opening of the patient's body. Cutting the sheath close to the human body can be risky since it may result in an inadvertent cut to the patient's body. Manipulation of the ureteroscope or other medical equipment a distance away from the body is inconvenient since excess movement could injure the patient at the access point of the equipment and since movement of the equipment at the proximal end of the sheath can result in movement at the distal end, where the medical procedure is performed. There is a need, therefore, for an adjustable length access sheath and a method to adjust the length of the access sheath after insertion into the patient's body.

### SUMMARY OF THE INVENTION

The invention is drawn to an adjustable length access sheath. The sheath has a distal end that is inserted into the patient, and a proximal end that extends from the patient. Once the distal end of the sheath is inserted to the desired length within the patient, the proximal end can be shortened. The invention consists of a number of preset, scored circumferential grooves that allow portions of the proximal end to be easily snapped off to the appropriate desired length.

### BRIEF DESCRIPTION OF THE DRAWINGS

FIG. 1 is a perspective view of a sheath with a dilator adapted for use with the sheath.

FIG. 2 is a perspective view of a sheath with the dilator operatively disposed within the prior art sheath.

FIG. 3 is an exploded perspective view of an end cap separate from a sheath.

FIG. 4 is a plan close-up of the protrusion end of a sheath showing the circumferential grooves.

FIG. 5 is a detail cross section of one embodiment of the boundary between the sheath and the protrusion end.

FIG. 6 is a detail cross section of a second embodiment of the boundary between the sheath and the protrusion end.

FIG. 7a is a side view of the invention before the length of the sheath has been adjusted.

FIG. 7b is a side view of the invention after the length of the sheath has been adjusted.

### DETAILED DESCRIPTION OF THE INVENTION

Detailed embodiments of the present invention are disclosed herein. It is to be understood that the disclosed embodiments are merely exemplary of the invention, and that there may be a variety of other alternate embodiments. The figures are not necessarily to scale, and some features may be exaggerated or minimized to show details of particular components. Therefore, specified structural and functional details disclosed herein are not to be interpreted as limiting, but merely as a basis for teaching one skilled in the art to employ the varying embodiments of the present invention.

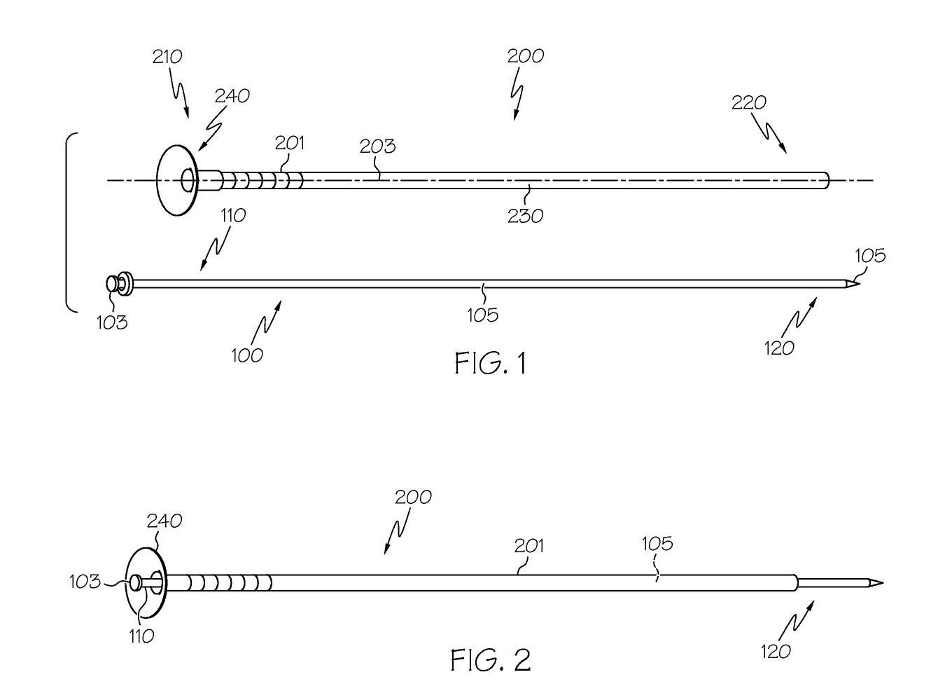

FIGS. 1 & 2 describe a typical sheath 200 with an internal dilator 100 or other medical equipment. An access sheath is illustrated in FIG. 1 and designated by reference number 200. In FIG. 1, the sheath 200 is illustrated in combination with a separate, but associated, dilator 100. The sheath 200 has the general configuration of an elongate tube 201 having an axis 203 which extends between a proximal end 210 and a distal end 220. A cap 240 is disposed at the proximal end 210 of the tube 201 and provides access into a working channel 230 which comprises the central bore of the tube 201. The dilator or other similar internal medical instrument 100 will typically have the configuration of an elongate rod 105 extending between a proximal end 110 and a distal end 120. A stopper 103 is disposed at the proximal end 110 to prevent the proximal end 110 of the dilator 100 from entering the sheath 200. A tapered tip 105 is formed at the distal end 120. The dilator 100 is adapted to be inserted into the working channel 230 of the sheath 200 with the distal end 120 extending distally of the sheath 200 with the tapered tip 105 extending from the distal end 220, and the stopper 103 extending proximally of the sheath 200, and preventing inadvertent insertion into the sheath 200. This operative position of the dilator 100 within the sheath 200 is illustrated in the assembled view of FIG. 2.

The sheath 200 is typically made from Teflon or other soft and flexible plastics that have good biocompatible properties for use within the human body, and also have low friction properties for ease of use within the body. Common examples of material used for such instruments include, but are not limited to, polyurethane, polyvinylchloride, natural or silicone rubbers, Teflon or other similar thermoplastics. Other acceptable materials include, but are not limited to medical grade PVC (polyvinyl chloride), Silicon coated latex, high-density polyethylene (HDPE), polytetrafluoroethylene (PTFE) (Teflon), PEBA (polyether block amide), PET (Polyethylene terephthalate), Polyolefin, polyurethane, polyimide, nylon, and thermoset and thermoplastics. Depending on the specific configuration and processing of these materials they can have properties that range from semi-rigid to very flexible. Some sheaths have an internal wire to provide a degree of rigidity, and to prevent bending or buckling during the insertion procedure. Other sheaths have an internal wire coil for the same purpose. In the preferred embodiment the sheath 200 is made from medical grade Teflon.

Dilators 100 come in a variety of outer diameter sizes, and can range from 8 French to 36 French. The most common sized dilator 100 for ureter access are between 10 or 13 French. A French is the standard measure of diameter of catheters, or other medical equipment, and each French is one third (⅓) of a millimeter. The internal diameter of the access sheath 200 needs to be just slightly larger than the outer diameter of the dilator 100 so that the dilator 100 can move easily and smoothly through the working channel 230 of the sheath 200. This means that the standard internal diameter of a sheath 200 is the equivalent of 10 to 13 French. The outer diameter of the sheath 200 is typically 2 French larger than the inner diameter. Typically sheaths and dilators are sold in paired sets where the first number is the diameter of the dilator and the second number is the outer diameter of the sheath, such as 9/11, 10/12, 11/13. So, for example, if the dilator 100 is 13 French the sheath 200 will be 15 French. This means that the wall 250 of the standard sheath 200 is the equivalent of 1 French, or one-third of a millimeter (0.33 mm) thick. The thickness of the sheath 200 can vary, and in some cases can be up to 2 mm in thickness for specialized procedures, but for common ureteral access sheaths 200 the wall 250 is approximately one-third of a millimeter (0.33 mm) thick.

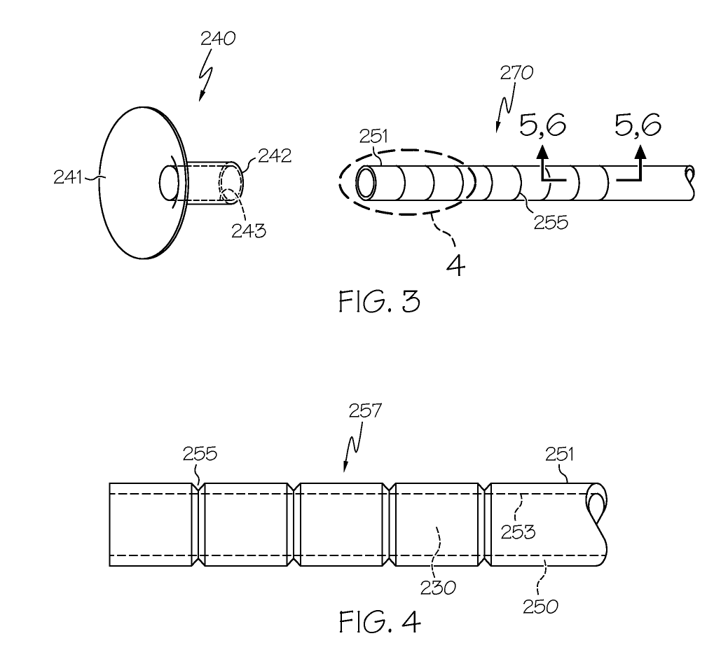

The tube 201 consists of an adjustable protrusion 270 located at the proximal end 210 of the sheath 200. As seen in FIG. 3, and in detail in FIG. 4, there are a series of grooves 255 along the protrusion 270, and a series of ribs 257 between the grooves. In the preferred embodiment the grooves 255 are spaced one centimeter apart. In the preferred embodiment the protrusion 270 is ten (10) centimeters long, creating a set of ten evenly spaced grooves 255. The grooves 255 are lateral to the axis 203 and circumferential around the tube 201. In alternate embodiments the grooves 255 can have different spacing, ranging from as close as one half a centimeter (0.5 cm) to perhaps one and one half centimeters (1.5 cm). In alternate embodiments the protrusion 270 can be longer than 10 centimeters, extending as much as 20 centimeters.

The tube 201 is defined by an elongated tubular wall 250 that has an outside tube face 251 and an inside tube face 253 which creates a wall thickness there-between. The wall thickness of the tubular wall 250, as defined above, is typically between 0.25 mm and 1.00 mm, but in the preferred embodiment is approximately 0.33 mm thick. The grooves 255 are scored circumferentially on the outside tube face 251, longitudinal to the axis 203. In most embodiments the tubular wall 250 is approximately 0.33 mm thick, and the groove 255 is between ¼ (one-quarter) and ⅔ (two-thirds) the thickness of the tubular wall 250, or between 0.22 mm and 0.08 mm. In the preferred embodiment the groove 255 will be one-half the wall thickness, so approximately 0.16 mm. The depth of the grooves 255 depend upon the stiffness or flexibility of the protrusion 270. As is well understood when scoring and snapping materials, the stiffer the protrusion 270 material, the shallower the grooves 255 must be to create a weak spot and allow the protrusion 270 to be snapped off.

The grooves 255 can be created during the molding process, or cut into the protrusion 270. Typically such elongated tubes are made by extraction molding or from a rolled sheet of material. In the preferred embodiment the grooves 255 are scored by rolling the protrusion 270 of the tube 201 while still hot and soft after the extrusion process. The protrusion 270 is rolled on a form with small spaced ridges which form the grooves 255. In one embodiment the tube 201 is made in a mold, and the grooves 255 are formed by the inclusion of spaced ridges within the mold. It is also possible to create the grooves 255 by cutting, or scoring, by rolling the tube 201 over a small cutting blade, or by rotating a cutting blade around the tube 201, similar to a tube cutting process well known in the plumbing trades.

FIG. 3 depicts the cap 240 separate from the sheath 200. The cap 240 has a cone shaped opening 241 and a mouth 242. The cone shaped opening 241 is cone shaped for ease of insertion of the dilator 100 or other medical instrumentation into the working channel 230. The mouth 242 has an inner diameter 243 that is sized to fit snuggly and securely onto the proximal end 210 of the sheath 200, and thus is equivalent to the outer diameter of the tube 201. The cap 240 is removably attached to the protrusion 270. The cap 240 is made of plastic with a slight degree of pliability to allow it to snuggly fit on and remain on the protrusion 270.

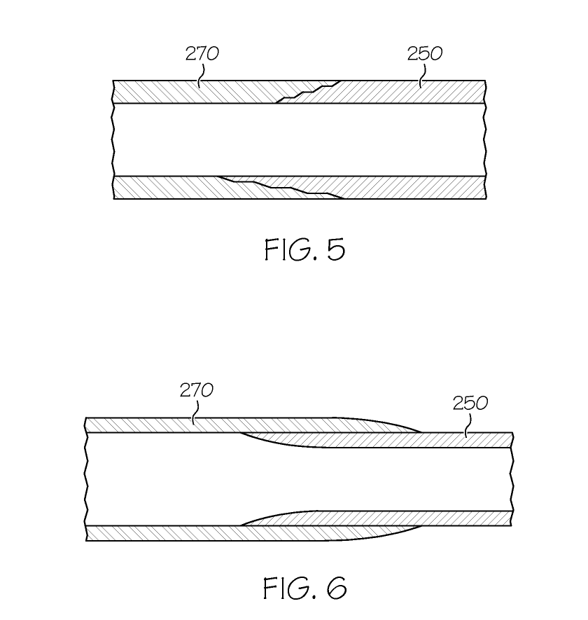

The protrusion 270 is tube shaped with a central bore coaxial with the working channel 230 of the sheath 200, and is integral with the tube 201. In one embodiment the protrusion 270 is made from the same material as the tube 201. The protrusion 270 must be stiffer than the tube 201 to allow it to be snapped off. The tube 201 has a first flexibility that allows it to be flexible enough to bend as appropriate during insertion into the human body. The protrusion 270 will have a second flexibility that is lower than the first flexibility of the tube 201. In some cases this second flexibility can be low enough to create a stiff or brittle protrusion 270. It is possible to create the protrusion 270 from the same material as the tube 201, but with less flexibility. This can be achieved in at least two common and well known ways; first by differential curing of the proximal end 210 of the tube 201, and second by the addition of various curing agents at the proximal end 210 to create a more stiff end. Differential curing is often achieved through the use of differing temperatures to speed up the solidification process and decrease flexibility. Common additives to decrease flexibility include various resins which decrease flexibility. In another embodiment the protrusion 270 is made from a different material having a lower degree of flexibility from the main component of the tube 201. In the preferred embodiment the protrusion 270 is made of a different, less flexible material, such as polyvinyl chloride (PVC), or other hard plastic or semi-rigid material, than the main portion of the tube 201. In this configuration the harder material can be extrusion molded at the end of the softer more flexible material to produce a sheath 200 with the protrusion 270 of less flexible material than the main portion of the tube 201. In one embodiment, depicted in FIG. 5, the protrusion 270 is adhered to and melted together with the tube 201 such that there is a uniform central bore 230 and outside tube face 251 diameter. In an alternate embodiment depicted in FIG. 6, the adjustable protrusion 270 is overlaid and adhered to the tube wall 250. In this configuration the protrusion 270 material is attached to the tube 201 material and adhered by melting or gluing or other suitable means. In this embodiment the protrusion 270 is essentially the same thickness as the tube 201, such that the outer diameter of the protrusion 270 is only slightly larger than the outer diameter of the sheath 200.

In use the combination sheath 200 and dilator 100 are inserted into the patient's body through the urethra. Depending upon the medical procedure, the sheath 200 and dilator 100 will be inserted to different positions within the body, which means that different lengths of the sheath 200 will extend from the body. In some procedures, such as catheterizing the bladder, the distal end 220 does not need to be precisely placed, but in other medical procedures, such as the removal of kidney stones, the distal end 220 needs to be positioned precisely. In order to achieve this goal, when a stone is in the ureter, it is preferable that the end of the sheath is 10-20 cm away from the stone. In the case where the stone is in the kidney proper, the proximal end of the sheath should be positioned as close to the junction of the ureter and kidney, without protruding into the kidney, which would make manipulation of the ureteroscope and thus visualization of the entire kidney, extremely difficult, if not impossible.

Once the sheath 200 and dilator 100 have been properly positioned inside the patient's body there will be a portion of the sheath 200 extending from the patient's urethra. At this point the dilator 100 will be removed from the sheath 200 to allow introduction of the specific medical device. The cap 240 can be removed from the proximal end 210, and the protrusion 270 can be adjusted to length.

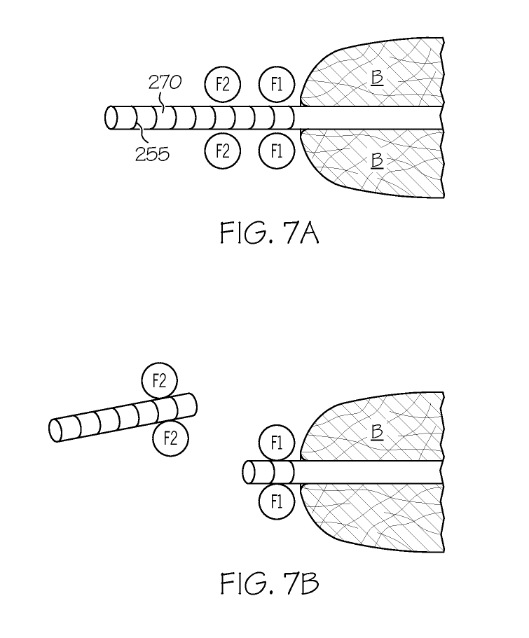

The process for adjusting the sheath 200 is described with reference to FIGS. 7a and 7b. As noted, the grooves 255 create weak spots in the protrusion 270. Because the protrusion 270 is at the proximal end of the sheath 200 it is not subject to twisting and other forces during the insertion process, so there is no possibility that the weak spots can cause a break in the sheath 200 during the initial insertion into the patient's body. However after proper placement of the sheath 200 and removal of the dilator 100 as described above, the grooves 255 allow the protrusion 270 to be adjusted to length. The doctor, or person performing the medical procedure, can grasp the sheath 200 at the desired location, and bend the sheath 200 with the other hand. As shown in FIG. 7a, the protrusion 270 will extend from the patient's body B, and the doctor will grasp the protrusion 270 near the body B with at least two fingers F1 of the first hand. This will stabilize the protrusion 270 to prevent the possibility of injury to the patient. This will also ensure that a section of the protrusion 270 will remain extending from the patient's body B for reattachment of the cap 240. The doctor will grasp the protrusion 270 on the other side of one of the grooves 255 with at least two fingers F2 of the other hand. The doctor will bend the protrusion 270 with the fingers F2 of the other hand with, while holding the protrusion 270 steady with the fingers F1 of the first hand. This torque or shear force, which will be essentially perpendicular to the axis 203 of the tube 201, will allow the tube 201 to snap off at the selected groove 255. The protrusion 270 can be adjusted in one centimeter lengths, so that the proper length of the sheath 200 can be achieved.

Once the tube 201 has been shortened to the desired length, the cap 240 can be reattached, and the appropriate medical equipment, such as a ureteroscope, can be inserted into the working channel 230 of the sheath 200, and the medical procedure performed.

The present invention is well adapted to carry out the objectives and attain both the ends and the advantages mentioned, as well as other benefits inherent therein. While the present invention has been depicted, described, and is defined by reference to particular embodiments of the invention, such reference does not imply a limitation to the invention, and no such limitation is to be inferred. The depicted and described embodiments of the invention are exemplary only, and are not exhaustive of the scope of the invention. Consequently, the present invention is intended to be limited only be the spirit and scope of the claims, giving full cognizance to equivalents in all respects.

I claim:

1. A method of adjusting the length of a ureteral access sheath comprising the steps of:
providing a sheath having a central bore, and outside wall, distal end and a proximal end;
scoring a series of spaced grooves in said proximal end of said outside wall;
inserting said sheath into a patient; and
adjusting the length of said sheath by breaking said proximal end at one of said grooves.

2. The method of adjusting the length of a ureteral access sheath of claim 1 comprising the further steps of:
disposing a dilator within said central bore of said sheath;
guiding said sheath with incorporated dilator into a patient to a desired location wherein a portion of said proximal end protrudes from said patient;
removing said dilator from said sheath; and
adjusting the length of said sheath by breaking said proximal end at one of said grooves near the body of said patient.

3. The method of adjusting the length of a ureteral access sheath of claim 2 comprising the further steps of:
removably attaching a cap to said proximal end of said sheath;
inserting a medical instrument into said central bore of said sheath to perform medical procedures within the body of the patient.

4. The method of adjusting the length of a uretral access sheath of claim 2 comprising the further steps of:
defining a wall thickness between said central bore and said outside wall, and
cutting said circumferential grooves into said outside wall to a depth of between one-quarter and two-thirds of said wall thickness.

5. The method of adjusting the length of a ureteral access sheath of claim 1 comprising the further steps of:
providing said sheath with a first flexibility;
operably disposing a protrusion at said proximal end of said sheath, said protrusion having an internal bore coaxial with said central bore, and said protrusion further having a second flexibility;
wherein said second flexibility is less than said first flexibility such that said grooves create a series of weak spots on said protrusion such that the length of said sheath can be adjusted by breaking said protrusion at one of said grooves.

6. The method of adjusting the length of a ureteral access sheath of claim 5 wherein the depth of said circumferential grooves is no more than two-thirds the wall thickness.

7. The method of adjusting the length of a ureteral access sheath of claim 5 wherein the depth of said circumferential grooves is no less than one-quarter the wall thickness.

8. The method of adjusting the length of a ureteral access sheath of claim 5 wherein said protrusion is made from a different material than said sheath, and wherein the material of the protrusion has less flexibility than the material of said sheath.

9. The method of adjusting the length of a ureteral access sheath of claim 1 wherein there are at least ten grooves spaced no more than one centimeter apart.

10. An adjustable length sheath comprising:
an elongated tube having a distal end, a proximal end, a central bore, and an outside wall;
said proximal end having a multiplicity of evenly spaced circumferential grooves cut into said outside wall, said grooves creating a weak point on said tube;
wherein said sheath is inserted into a patient's body to a desired position and wherein the length of said tube can be adjusted by selecting one of said multiplicity of said grooves to the desired length and snapping said tube at said groove.

11. The adjustable length sheath of claim 10 further comprising;
a dilator or other medical device operably disposed within said central bore of said sheath;
a conical cap removably attached to said proximal end; and
wherein said sheath is inserted into the patient to a medically appropriate position thus leaving a portion of said sheath extending from the patient, and wherein the length of said sheath can be adjusted by breaking said tube at one of said grooves.

12. The adjustable length sheath of claim 10 further comprising;
a protrusion having a second flexibility and wherein said sheath has a first flexibility and wherein said second flexibility is less than said first flexibility such that said protrusion is more rigid than said sheath, and wherein said grooves are cut into said protrusion such that the protrusion can be easily broken at one of said grooves to alter the length of said sheath.

13. The adjustable length sheath of claim 12 wherein said protrusion is made from a different material than said sheath, said different material having less flexibility than the material of said sheath.

14. The adjustable length sheath of claim 10 further comprising;
a wall thickness defined between said central bore and said outside wall, and wherein said grooves are cut to between one quarter and two thirds of the thickness of said wall thickness.

15. The adjustable length sheath of claim 10 wherein said protrusion is between five (5) centimeters and twenty (20) centimeters in length.

16. The adjustable length sheath of claim 10 wherein said grooves are evenly spaced between one half (0.5) and one and a half (1.5) centimeters apart.

17. An adjustable length uretral access sheath comprising:
an elongated tube having a distal end, a proximal end, a central bore, and an outside wall;
a dilator sized and configured for workable insertion within said central bore;
said proximal end having an adjustable protrusion, said adjustable protrusion having a lower flexibility than said tube and a multiplicity of evenly spaced circumferential grooves scored around the outside wall of said protrusion, wherein said grooves produce a multiplicity of weak spot in said protrusion;
a removably attachable conical cap sized for removable attachment to said adjustable protrusion;
wherein said dilator is inserted into said central bore of said tube and said tube and dilator are inserted into a patient's body to a desired position such that the adjustable protrusion protrudes from said patient's body, said dilator removed from said central bore, said conical cap removed from said adjustable protrusion, and the length of said sheath adjusted by snapping said adjustable protrusion at a desired groove, and wherein further said conical cap is replaced on said adjustment protrusion and said dilator or other desired medical instrument inserted into said central bore of said tube.
